(19) United States
(12) Patent Application Publication
(10) Pub. No.: US 2020/0229884 A1
(43) Pub. Date: Jul. 23, 2020

(54) Medical Glove

(71) Applicant: TONY KIAN DJIE, Alhambra, CA (US)

(72) Inventor: TONY KIAN DJIE, Alhambra, CA (US)

(21) Appl. No.: 16/254,399

(22) Filed: Jan. 22, 2019

(51) Int. Cl. A61B 42/10; A41D 19/00

(52) U.S. Cl. CPC A61B 42/10; A41D 19/0082; A41D 2500/50; A41D 19/0075; A41D 2600/20; A41D 19/0062

(57) ABSTRACT

A medical glove includes a glove body and a biohazard indicator. The glove body, which is a disposable glove, has an outer surface and an inner surface, wherein the outer surface of the glove body is contaminated after it is used. The biohazard indicator is provided at the inner surface of the glove body, wherein when the glove body is donned, the biohazard indicator is hidden underneath the outer surface of the glove body. When the glove body is doffed to flip the glove body inside-out, the inner surface of the glove body wraps the outer surface thereof to expose the biohazard indicator for indicating the glove body being used and contaminated.

### BACKGROUND

### 1. Field of the Invention

The present invention primarily relates to a medical apparatus. Particularly, the present invention relates to a medical glove which has a biohazard indicator being hidden when the medical glove is donned and is indicated when the medical glove is doffed.

### 2. Discussion of the Related Art

Medical gloves are essential to medical professionals, such as nurse, laboratorian, and surgeon, being used during medical examinations and procedures to protect themselves from possible infection when their hands touch any harmful substances. Once the gloves are used, the used gloves are considered as medical wastes or biohazardous wastes which contain harmful microorganisms that can infect patients and/or medical professionals. Therefore, the used gloves must be properly thrown away due to the potential for contamination. Generally speaking, the used gloves are disposed in a closable biohazard bag, so that a medical waste handling operator will transport the biohazard bag to the medical waste management company. However, if the used gloves are not properly thrown in the biohazard bag, it is hard to know whether the gloves are being used or not. Some patients, such as young child, may unintentionally touch the gloves left on the physician's desk, or the medical waste handling operator may pick up the gloves accidentally dropped on the floor. It is always a concern of how to protect people from risks related to medical waste.

Accordingly, it would be highly desirable to develop a medical glove which is capable of letting people know the glove as the medical waste after being used in order to handle the glove free from anxiety.

### BRIEF SUMMARY OF THE INVENTION

According to the present invention, a medical glove is described. The medical glove comprises a biohazard indicator to indicator the medical glove being used and contaminated after it is doffed.

According to the present invention, the contaminated outer surface of the glove body is wrapped within the clean inner surface thereof after it is doffed to prevent any potential contamination after the use of the medical glove.

It is another object of the present invention to provide a medical glove, wherein the biohazard indicator is hidden when the medical glove is donned.

It is further another object of the present invention to provide a medical glove, which permits the user to quickly and easily notify any used medical glove via the biohazard indicator once the medical glove is doffed.

It is further another object of the present invention to provide a medical glove, wherein the biohazard indicator can be incorporated with any type of conventional medical glove such as surgical glove.

It is yet another object of the present invention to provide a medical glove, wherein the biohazard indicator is provided inside the glove body in a hidden manner to let the wearer using the glove free from anxiety.

It is yet another object of the present invention to provide a medical glove which is lightweight, disposable, easy to use without assistance, and inexpensive to manufacture.

It is yet another object of the present invention to provide a medical glove, wherein the biohazard indicator can be provided on any material of the glove body.

For a more complete understanding of the present invention with its objectives and distinctive features and advantages, reference is now made to the following specification and to the accompanying drawings.

### BRIEF DESCRIPTION OF THE DRAWING(S)

These and other features, aspects and advantages of the present invention will become better understood from the following description, appended claims, and accompanying figures where:

FIG. 1 is a front view of a medical glove according to an embodiment of the present invention.

FIG. 2 is a rear view of the medical glove according to the present invention.

FIG. 3 is a front view of the medical glove according to the present invention, showing the medical glove being flipped inside-out to expose the biohazard indicator.

FIG. 4 is a rear view of the medical glove according to the present invention, showing the medical glove being flipped inside-out to expose the biohazard indicator.

FIG. 5 shows a pair of doffed medical gloves with the biohazard indicator.

FIG. 6 shows an alternative mode of the biohazard indicator on the inner side of the glove body according to the present invention.

FIG. 7 shows another alternative mode of the biohazard indicator on the inner side of the glove body according to the present invention.

FIG. 8 is a flow diagram of a manufacturing method of the medical glove according to the present invention.

### DETAILED DESCRIPTION OF THE INVENTION

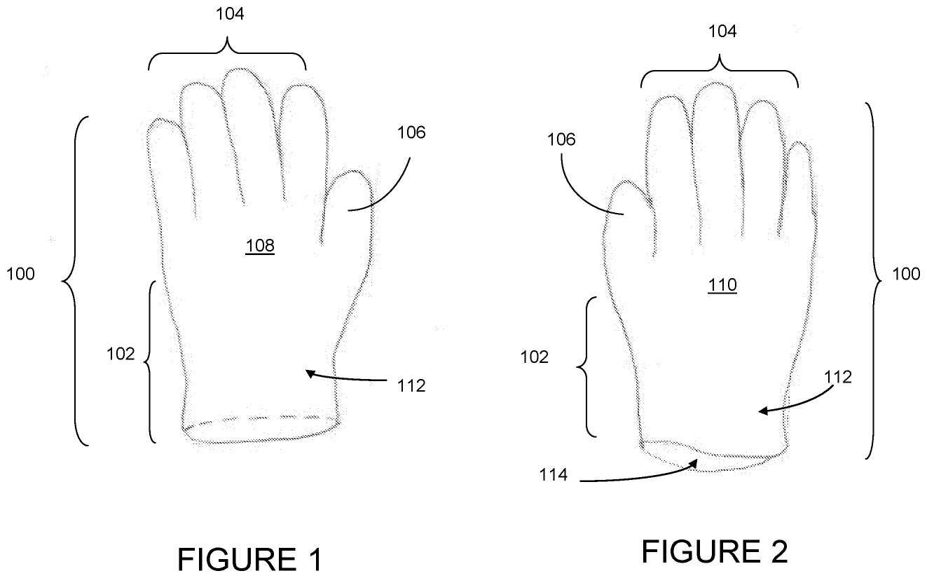

For purposes of description herein the terms “upper,” “lower,” “right,” “left,” “rear,” “front,” “vertical,” “horizontal” and derivatives thereof shall relate to the invention as oriented in FIGS. 1 and 2. However, it is to be understood that the invention may assume various alternative orientations and step sequences, except where expressly specified to the contrary. It is also to be understood that the specific devices and processes illustrated in the attached drawings, and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

As used in this disclosure, the term “comprise” and variations of the term, such as “comprising” and “comprises,” are not intended to exclude other additives, components, integers or steps.

The present invention provides a medical glove according to an embodiment is disclosed. The medical glove is used during medical examinations and procedures to protect the wearers from possible infection. It should be appreciated that the following distinctive features can be applied to any non-medical glove as long as the glove must be notified as a used glove after it is doffed. The glove can be formed in one or in pair.

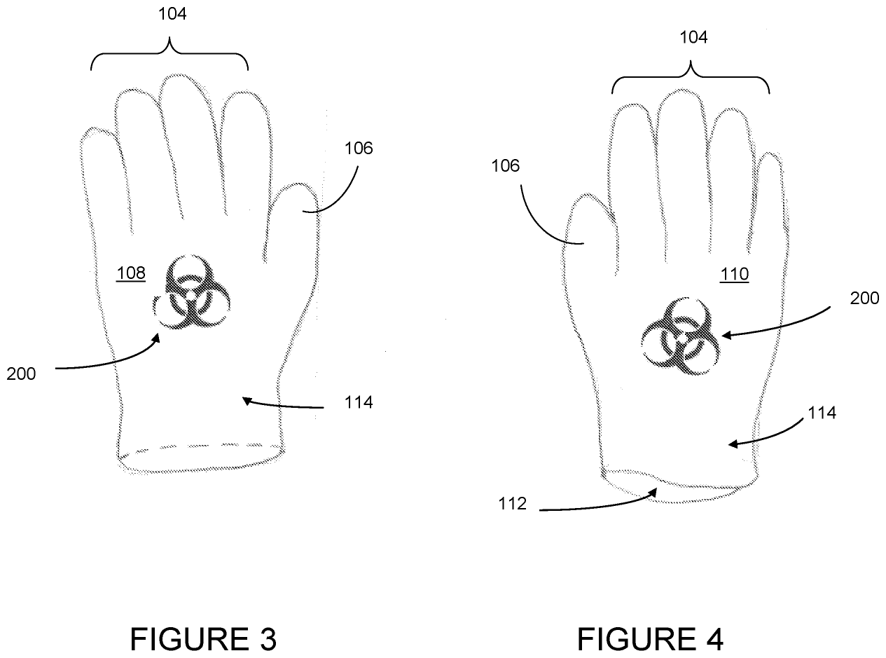

Reference is now made to certain specific non-limiting embodiment as shown in Figures. FIGS. 1 and 2 depict the front and rear views of the medical glove respectively. FIGS. 3 and 4 depict the front and rear views of the medical glove being flipped inside-out respectively. The medical glove comprises a glove body 100 and a biohazard indicator 200.

The glove body 100 is a disposable glove made of high strength and disposable material. The glove body 100 can be made of natural rubber, vinyl, Chloroprene, or Isoprene. It is preferred that the glove body 100 is made of Nitrile (Butadiene). The glove body 100 has a hand portion 102, four finger portions 104 and a thumb portion 106, and defines a glove cavity for receiving a wearer's hand. Thus, the glove body 100 comprises a backhand layer section 108 and a palm layer section 110 to form the hand portion 102 therebetween. Therefore, the glove body 100 is formed to have a general contour of the wearer's hand for the wear to don the glove body 100.

According to the present invention, the glove body 100 has an outer surface 112 and an inner surface 114 as an opposite side of the outer surface 112, wherein the outer surface 112 and the inner surface 114 are made of same material, so that the two surfaces of the glove body 100 are the outer surface 112 and the inner surface 114 respectively. In other words, the glove body 100 is made of a single layer structure to define the outer surface 112 and the inner surface 114 at two opposite surfaces of the single layer structure respectively. The single layer structure of the glove body 100 includes the backhand layer section 108 and the palm layer section 110 integrated together to form the hand portion 102.

When the glove body 100 is donned, the inner surface 112 of the glove body 100 is arranged for contacting a wearer's skin. When the glove body 100 is doffed, the glove body 100 is flipped inside-out as shown in FIGS. 3 and 4. The outer surface 112 of the glove body 100 is a working surface configured for contacting a contaminated object. The inner surface 114 of the glove body 100 is a non-working surface not supposed for contacting any contaminated object. Therefore, the glove body 100 is a non-reversible wearing glove. Accordingly, the outer surface 112 of the glove body 100 is contaminated after it is used. Once the glove body 100 is doffed, the contaminated outer surface 112 is wrapped inside the cleaned inner surface 114 for prevent any potential contamination for the used glove body 100.

FIGS. 3 and 4 depict the biohazard indicator 200 provided at the inner surface 114 of the glove body 100. When the glove body is donned, the biohazard indicator 200 is hidden underneath the outer surface 112 of the glove body 100, as shown in FIGS. 1 and 2. When the glove body is doffed to flip the glove body 100 inside-out, as shown in FIGS. 3 and 4, the outer surface 112 of the glove body 100 is wrapped within the inner surface 114 thereof to expose the biohazard indicator 200 for indicating the glove body 100 being used and contaminated. Since the glove body 100 is a non-reversible wearing glove, the wearer will not flip the glove body 100 inside-out after it is donned. Therefore, the biohazard indicator 200 will be seen only when the glove body 100 is doffed.

Referring now to FIGS. 3 and 4, the biohazard indicator 200 comprises a biohazard symbol formed at the inner surface 114 of the glove body 100. The biohazard symbol is a well known symbol used as a warning symbol in the labeling of biological materials with potential health risk. People will pay more attention and be caution when an object is labeled with the biohazard symbol. As the biohazard symbol is formed at the inner surface 114 of the glove body 100, the biohazard symbol of the biohazard indicator 200 is invisible through the outer surface 112 of the glove body 100. Therefore, when the glove body 100 is donned, the biohazard indicator 200 cannot be seen to let the wearer using the glove body 100 free from anxiety.

According to the present invention, the biohazard symbol of the biohazard indicator 200 is formed on one of the backhand layer section 108 and the palm layer section 110 at the inner surface 114 of the glove body 100. Preferably, the biohazard symbol of the biohazard indicator 200 is formed on both the backhand layer section 108 and the palm layer section 110 at the inner surface 114 of the glove body 100.

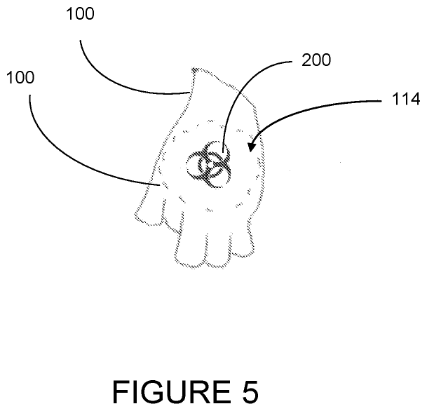

It should be appreciated that the wearer wears a pair of medical gloves during medical examinations and procedures as an example. There is a guide for doffing the medical gloves by the steps of peeling downward one glove body 100, for example the left handed glove, from the left wrist of the wearer and turning the left glove body 100 inside-out until the left glove body 100 is removed from the left hand of the wearer. Therefore, the contaminated outer surface 112 of the left glove body 100 is wrapped within the inner surface 114 thereof to expose the biohazard indicator 200 at the left glove body 100. Then, the left handed glove 100 is held by the gloved hand (right hand). With the un-gloved hand (left hand), the right glove body 100 is pulled downward from the right wrist of the wear and is turned inside-out until the right glove body 100 is removed from the right hand of the wearer. Therefore, the left glove body 100 is enveloped inside the right glove body 100 with no contaminant on both hands of the wearer. Thus, the contaminated outer surface 112 of the right glove body 100 is wrapped within the inner surface 114 thereof to expose the biohazard indicator 200 at the right glove body 100 as shown in FIG. 5.

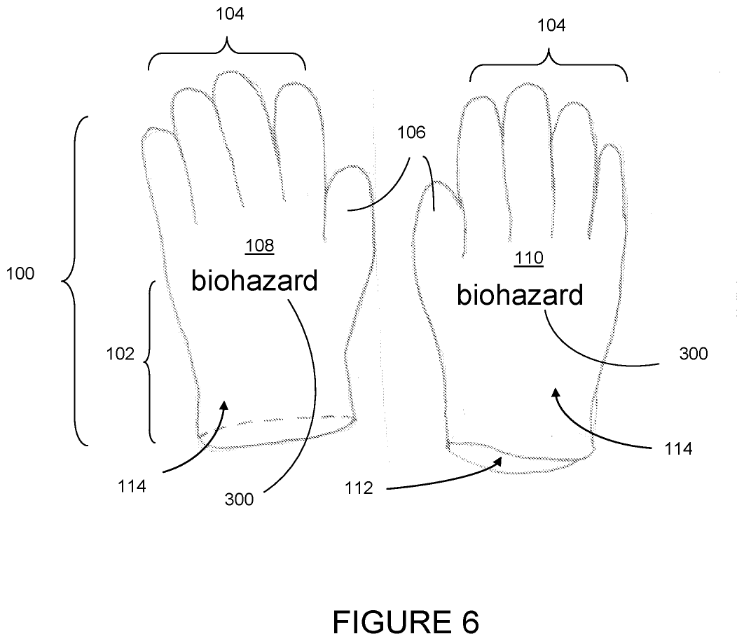

Referring now to FIG. 6, an alternative mode of the biohazard indicator 300 is illustrated. The biohazard indicator 300 comprises a word of “biohazard” formed at the inner surface 114 of the glove body 100. As the biohazard word of the biohazard indicator 300 is formed at the inner surface 114 of the glove body 100, the biohazard word of the biohazard indicator 300 is invisible through the outer surface 112 of the glove body 100. Therefore, when the glove body 100 is donned, the biohazard indicator 300 cannot be seen.

The biohazard word of the biohazard indicator 300 is formed on one of the backhand layer section 108 and the palm layer section 110 at the inner surface 114 of the glove body 100. Preferably, the biohazard word of the biohazard indicator 300 is formed on both the backhand layer section 108 and the palm layer section 110 at the inner surface 114 of the glove body 100. It should be appreciated that the biohazard indicator 300 can be any warning words, such as “DO NOT USE”, “USED”, DO NOT REUSE″, “WARNING”, “CONTAMINATED”, SOILED”, “DISPOSE”, “THROW AWAY”, “SINGLE USE”, “SINGLE USE ONLY”, and the like.

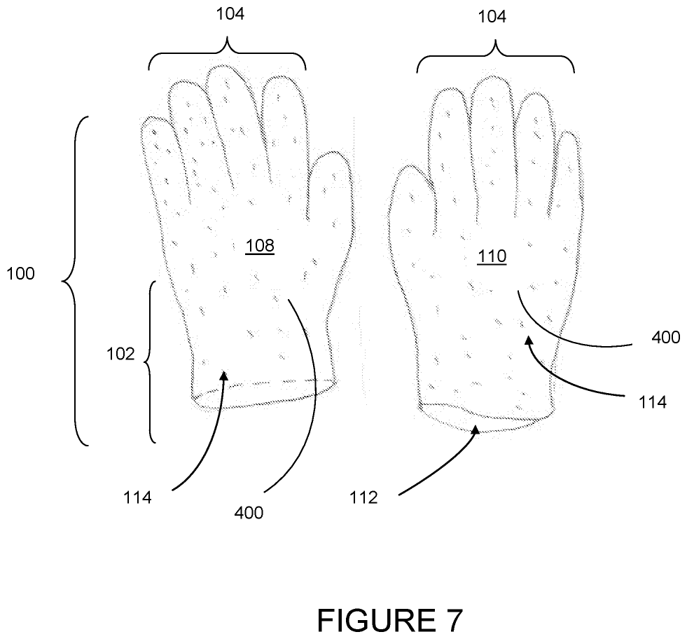

Referring now to FIG. 7, another alternative mode of the biohazard indicator 400 is illustrated. The color of the inner surface 114 of the glove body 100 is different from the color of the outer surface 112 of the glove body, wherein the biohazard indicator 400 is the color of the inner surface 114 of the glove body 100. When the glove body 100 is donned, the color of the inner surface 114 thereof cannot be seen, so that the biohazard indicator 400 is invisible through the outer surface 112 of the glove body 100. It is worth mentioning that since the glove body 100 is a non-reversible wearing glove, the color of the inner surface 114 of the glove body 100 cannot be seen unless the glove body 100 is inside-out. When the glove body 100 is doffed, the glove body 100 is flipped inside-out, so that the biohazard indicator 400 will be exposed via the color of the inner surface 114 of the glove body 100. The color of the inner surface 114 of the glove body 100 is preferably yellow or red.

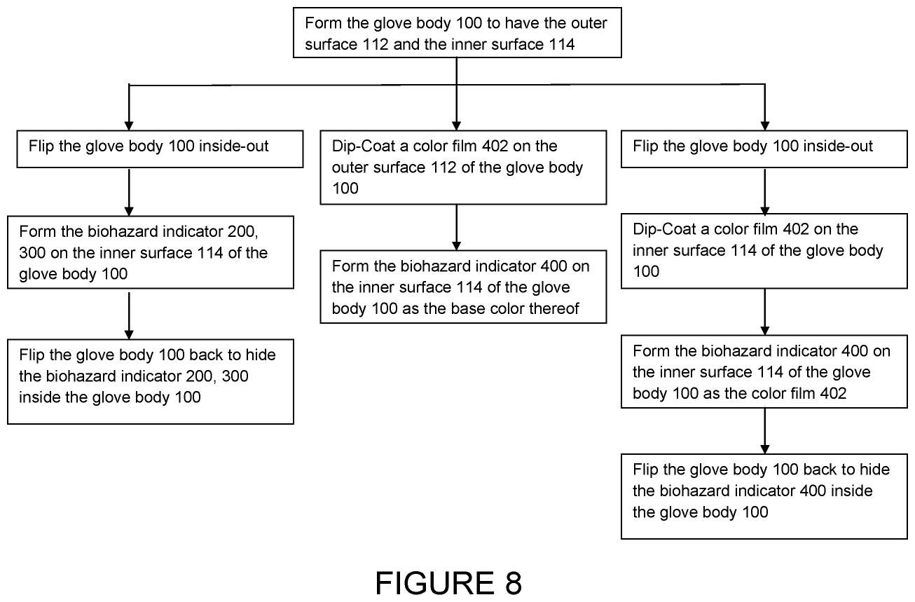

Referring now to FIG. 8, the present invention further provides a manufacturing method of the medical glove. The manufacturing method of the medical glove is simple and inexpensive. The manufacturing method comprises the following steps.

(1) Form the glove body 100 to have the outer surface 112 and the inner surface 114. The glove body 100 can be made by a conventional manufacturing process to have a general contour of the wearer's hand.

(2) Form the biohazard indicator 200, 300, 400 at the inner surface 114 of the glove body 10. Accordingly, there are different ways to form the biohazard indicator 200, 300, 400 at the inner surface 114 of the glove body 10.

In order to form the biohazard indicator 200, 300 of the preferred embodiment, after the step (1) and before the (2), the method further comprises a step of flipping the glove body 100 inside-out. As a result, the inner surface 114 of the glove body 100 is flipped out that the outer surface 112 thereof is flipped inside the glove body 100. Therefore, the biohazard symbol of the biohazard indicator 200 and/or the biohazard word of the biohazard indicator 300 can be formed on the inner surface 114 of the glove body 100. It is preferred that the biohazard symbol of the biohazard indicator 200 and/or the biohazard word of the biohazard indicator 300 can be printed or coated on the inner surface 114 of the glove body 100 at least one of the backhand layer section 108 and the palm layer section 110 of the glove body 100. Then, after the step (2), the method further comprises a step of flipping back the glove body 100 so that the inner surface 114 of the glove body 100 is flipped back inside to hide the biohazard symbol of the biohazard indicator 200 and/or the biohazard word of the biohazard indicator 300 inside the glove body 100.

Alternatively, in order to form the biohazard indicator 400, the method, in the step (2), further comprises a step of dip-coating a color film 402 on the glove body 100. It should be appreciated that the glove body 100 has a base color when the glove body 100 is made in the step (1). Therefore, the color film 402 is coated on the outer surface 112 of the glove body 100, wherein the color of the color film 402 is different from the base color of the glove body 100. As a result, the color of the inner surface 114 of the glove body 100 is the same color of the glove body 100 and is different from the color of the color film 402. The base color of the glove body 100 is configured to be the biohazard indicator 400. It should be appreciated that the base color can be selectively altered by adding a desired color agent, such as red or yellow, into the material of the glove body 100 when manufacturing the glove body 100 in the step (1).

Alternatively, in order to form the biohazard indicator 400, after the step (1) and before the (2), the method further comprises a step of flipping the glove body 100 inside-out. As a result, the inner surface 114 of the glove body 100 is flipped out that the outer surface 112 thereof is flipped inside the glove body 100. Then, the method, in the step (2), further comprises a step of dip-coating a color film 402 on the inner surface 114 of the glove body 100. So that the color film 402 is coated on the inner surface 112 of the glove body 100, wherein the color of the color film 402 is different from the base color of the glove body 100. As a result, the color of the outer surface 112 of the glove body 100 is the same color of the glove body 100 and is different from the color of the color film 402. The color of color film 402 is configured to be the biohazard indicator 400. It should be appreciated that the color film 402 can be selectively altered by adding a desired color agent, such as red or yellow, in order to coat on the inner surface 112 of the glove body 100 to form the biohazard indicator 400. Then, after the step (2), the method further comprises a step of flipping back the glove body 100 so that the inner surface 114 of the glove body 100 is flipped back inside to hide the inner surface 113 of the glove body 100 as the biohazard indicator 400 inside the glove body 100.

While the embodiments and alternatives of the invention have been shown and described, it will be apparent to one skilled in the art that various other changes and modifications can be made without departing from the spirit and scope of the invention.

What is claimed is:

1. A medical glove, comprising:
a glove body, which is a disposable and non-reversible wearing glove, having an outer surface and an inner surface, wherein said glove body is made of a single layer structure to define said outer surface and said inner surface at two opposite surfaces of said single layer structure respectively, wherein said outer surface of said glove body is considered to be contaminated after it is used; and
a biohazard indicator provided at said inner surface of said glove body, wherein when said glove body is donned, said biohazard indicator is hidden underneath said outer surface of said glove body, wherein when said glove body is doffed to flip said glove body inside-out, said inner surface of said glove body wraps said outer surface thereof to expose said biohazard indicator for indicating said glove body being used and contaminated.

2. The medical glove of claim 1, wherein said biohazard indicator comprises a biohazard symbol formed at said inner surface of said glove body.

3. The medical glove of claim 2, wherein said biohazard symbol of said biohazard indicator is invisible through said outer surface of said glove body.

4. The medical glove of claim 2, wherein said glove body comprises a backhand layer section and a palm layer section to form a hand portion, wherein said biohazard symbol is formed on one of said backhand layer section and said palm layer section at said inner surface of said glove body.

5. The medical glove of claim 2, wherein said glove body comprises a backhand layer section and a palm layer section to form a hand portion, wherein said biohazard symbol is formed on both said backhand layer section and said palm layer section at said inner surface of said glove body.

6. The medical glove of claim 1, wherein said biohazard indicator comprises a word of biohazard formed at said inner surface of said glove body.

7. The medical glove of claim 6, wherein said biohazard word of said biohazard indicator is invisible through said outer surface of said glove body.

8. The medical glove of claim 6, wherein said glove body comprises a backhand layer section and a palm layer section to form a hand portion, wherein said biohazard word is formed on one of said backhand layer section and said palm layer section at said inner surface of said glove body.

9. The medical glove of claim 6, wherein said glove body comprises a backhand layer section and a palm layer section to form a hand portion, wherein said biohazard word is formed on both said backhand layer section and said palm layer section at said inner surface of said glove body.

10. The medical glove of claim 1, wherein color of said inner surface of said glove body is different from color of said outer surface of said glove body, wherein said biohazard indicator is said color of said inner surface of said glove body.

11. The medical glove of claim 10, wherein said color of said inner surface of said glove body is yellow or red.

12. The medical glove of claim 10, further comprising a color film dip-coated on said inner surface of said glove body, wherein said color of said inner surface of said glove body is a base color of said glove body.

13. The medical glove of claim 10, further comprising a color film dip-coated on said inner surface of said glove body to form said biohazard indicator.

14. The medical glove of claim 1, wherein said glove body is made of Nit (Butadiene).

15. The medical glove of claim 1, wherein said glove body is made of material selected from a group consisting of natural rubber, vinyl, Chloroprene, and Isoprene.

16. A method of manufacturing a medical glove, comprising the steps of:
(a) a forming a disposable glove body in a single layer structure to have an outer surface and an inner surface, wherein said glove body is formed to have a general contour of a wearer's hand, wherein said outer surface of said glove body is considered to be contaminated after it is used; and
(b) forming a biohazard indicator at said inner surface of said glove body, wherein when said glove body is donned, said biohazard indicator is hidden underneath said outer surface of said glove body, wherein when said glove body is doffed to flip said glove body inside-out, said inner surface of said glove body wraps said outer surface thereof to expose said biohazard indicator for indicating said glove body being used and contaminated.

17. The method of claim 16, before the step (b), further comprising a step of flipping said glove body inside-out to expose said inner surface thereof in order to coat said biohazard indicator, and after the step (b), further comprising a step of flipping said glove body back to hide said biohazard indicator underneath said outer surface of said glove body.

18. The method of claim 16, wherein said biohazard indicator is one of a biohazard sign and a word of biohazard provided on said inner surface of said glove body.

19. The method of claim 16, in the step (b), further comprising a step of dip-coating a color film on said glove body to configure a color of said inner surface of said glove body being different from a color of said outer surface of said glove body, wherein said biohazard indicator is said color of said inner surface of said glove body.

20. The method of claim 16, before the step (b), further comprising a step of flipping said glove body inside-out to expose said inner surface thereof in order to dip-coat a color film on said glove body, and after the step (b), further comprising a step of flipping said glove body back, such that color of said inner surface of said glove body is different from color of said outer surface of said glove body, wherein said biohazard indicator is said color of said inner surface of said glove body.
